(19) United States
(12) Patent Application Publication
(10) Pub. No.: US 2019/0255023 A1
(43) Pub. Date: Aug. 22, 2019

(54) COMBINATION THERAPY COMPRISING A THIAZOLE AND A CORTICOSTEROID TO TREAT SKIN CONDITIONS

(71) Applicant: Avexxin AS, Trondheim (NO)

(72) Inventors: Berit Johansen, Trondheim (NO); Astrid Jullumstrø Feuerherm, Trondheim (NO)

(21) Appl. No.: 16/347,253

(22) PCT Filed: Nov. 3, 2017
(86) PCT No.: PCT/EP2017/078162

§ 371 Date: May 3, 2019

(30) Foreign Application Priority Data
Nov. 4, 2016 (GB) 1618647.0

(51) Int. Cl. A61K 31/426; A61K 31/573; A61P 17/04; A61P 17/06; A61K 9/00; A61K 9/06; A61K 9/12

(52) U.S. Cl. CPC A61K 31/426; A61K 31/573; A61P 17/04; A61K 9/122; A61K 9/0014; A61K 9/06; A61P 17/06

(57) ABSTRACT

A pharmaceutical composition comprising: (A) at least one compound of formula (I): wherein X is O or S, preferably O R₆ is H, C₁₋₆alkyl, —(CH₂)pCOOH, —(CH₂)ₚCOOC₁₋₆alkyl, —(CH₂)ₚCONH₂, —(CH₂)ₚCONHC₁₋₆alkyl, —(CH₂)ₚCON(C₁₋₆alkyl)₂, R¹¹ is H or C₁₋₆ alkyl; each R₅ is —OC₁₋₁₀alkyl, —SC₁₋₁₀alkyl, —C₁₋₁₂alkyl, or OAr²; wherein Ar² is phenyl, optionally substituted with one or more halo; each p is 0 to 3; each z is 1 to 2; or a pharmaceutically acceptable salt, or a hydrate or solvate thereof; and (B) one or more corticosteroid partners, preferably selected from the group consisting of betamethasone, clobetasol, halometasone, dexamethasone, fluocortolone, desoximetasone, diflorasone, fluocinonide, flurandrenolide, halobetasol, amcinonide, halocinonide, triamcinolone, hydrocortisone, aclometasone, fluticasone, mometasone, clocortolone, fluocinolone, desonide, prednisone, prednisolone, and prednicarbate or a pharmaceutically acceptable salt, or a hydrate or solvate thereof, especially betamethasone or a pharmaceutically acceptable salt, or a hydrate or solvate thereof.

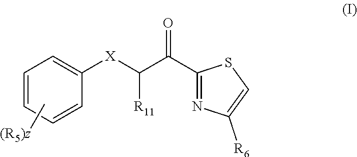

This invention relates to a pharmaceutical composition comprising certain thiazole derivatives in combination with certain corticosteroids such as betamethasone or a pharmaceutically acceptable salt, or a hydrate or solvate thereof. The invention also relates to the use of said pharmaceutical composition for the treatment or prevention of skin conditions such as dermatitis and psoriasis.

### BACKGROUND

This invention is concerned with a combination therapy for the treatment of certain skin conditions such as psoriasis and dermatitis. In its broadest sense, dermatitis is inflammation of the skin. It is a common and disfiguring skin condition which requires quick and efficient treatment. Dermatitis symptoms vary, however, with the different forms of the condition. Symptoms vary from skin rashes to bumpy rashes through to flaky skin and blisters. Although different types of dermatitis have varying symptoms, there are certain signs that are common for all of them, including redness of the skin, swelling, itching, skin lesions and sometimes oozing and scarring.

Also, the area of the skin on which the symptoms appear tends to be different with every type of dermatitis. Types of dermatitis are classified according to the cause of the condition. Contact dermatitis is caused by an allergen or an irritating substance. Irritant contact dermatitis accounts for 80% of all cases of contact dermatitis.

Atopic dermatitis is very common worldwide and increasing in prevalence. Atopic dermatitis is a type of eczema and is an inflammatory, chronically relapsing, non-contagious and itchy skin disorder.

Other less common forms of dermatitis include dermatitis herpetiformis. It is characterized by intensely itchy, chronic papulovesicular eruptions, usually distributed symmetrically on extensor surfaces such as the back of neck, scalp, elbows, knees, back, hairline, groin or face.

Seborrheic dermatitis is a dermatitis that occurs in the vicinity of sebaceous glands and is caused by sebum over production. The condition tends to give a scaly, flaky skin condition.

Stasis dermatitis is an inflammation on the lower legs which is caused by build-up of blood and fluid and it is more likely to occur in people with varicose veins.

Other common skin disorders include psoriasis. This is an autoimmune induced, chronic disease of skin characterised by red, itchy and scaly skin patches. Skin disorders in general and dermatitis and psoriasis in particular are disfiguring and can lead to reluctance of a sufferer to let people see their condition. Successful treatments of these skin disorders are therefore sought.

A common treatment for skin disorders is administration of one or more topical corticosteroids. The present inventors have now found that the combination of certain thiazoles and certain corticosteroids such as betamethasone or a pharmaceutically acceptable salt, or a hydrate or solvate results in a synergistic improvement in performance.

### SUMMARY OF INVENTION

Thus, viewed from one aspect the invention provides a pharmaceutical composition comprising:

(A) at least one compound of formula (I):

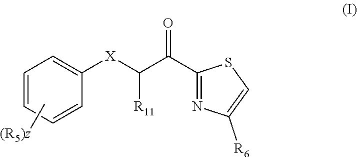

wherein X is O or S, preferably O

R₆ is H, C₁₋₆alkyl, —(CH₂)ₚCOOH, —(CH₂)ₚCOOC₁₋₆alkyl, —(CH₂)ₚCONH₂, —(CH₂)ₚCONHC₁₋₆alkyl, and —(CH₂)ₚCON(C₁₋₆alkyl)₂;

R¹¹ is H or C₁₋₆ alkyl;

each R₅ is —OC₁₋₁₀alkyl, —SC₁₋₁₀alkyl, —C₁₋₁₂alkyl, or OAr²;

wherein Ar² is phenyl, optionally substituted with one or more halo;

each p is 0 to 3;

each z is 1 to 2;

or a pharmaceutically acceptable salt, or a hydrate or solvate thereof; and

(B) one or more corticosteroid partners, preferably selected from the group consisting of betamethasone, clobetasol, halometasone, dexamethasone, fluocortolone, desoximetasone, diflorasone, fluocinonide, flurandrenolide, halobetasol, amcinonide, halocinonide, triamcinolone, hydrocortisone, aclometasone, fluticasone, mometasone, clocortolone, fluocinolone, desonide, prednisone, prednisolone and prednicarbate or a pharmaceutically acceptable salt, or a hydrate or solvate thereof.

In a preferred embodiment, betamethasone or a pharmaceutically acceptable salt, or a hydrate or solvate thereof is the corticosteroid partner.

Viewed from another aspect the invention provides a pharmaceutical kit composition for simultaneous, in parallel, sequential or separate use comprising a first composition comprising at least one compound (I) as herein defined and a pharmaceutically-acceptable diluent or carrier, and a second composition comprising at least one compound (B) as the corticosteroid partner as herein defined such as betamethasone or a pharmaceutically acceptable salt, or a hydrate or solvate thereof and a pharmaceutically-acceptable diluent or carrier.

In particular, the invention relates to a pharmaceutical composition, or kit as herein before defined in which the compound of formula (I) is:

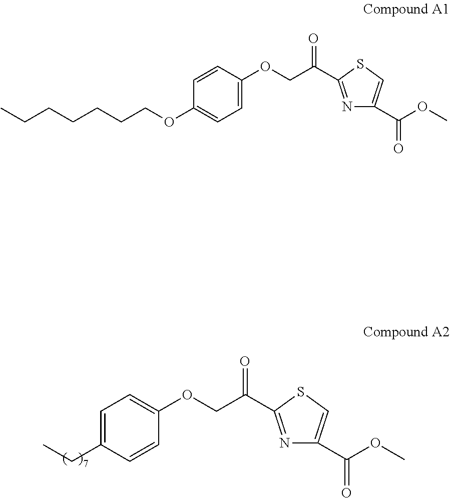

or a pharmaceutically acceptable salt, or a hydrate or solvate thereof. In particular, the corticosteroid partner (B) is betamethasone or a pharmaceutically acceptable salt, or a hydrate or solvate thereof.

At least one other corticosteroid partner may be combined with the betamethasone to achieve intended results, for example, 1 or 2 of such compounds. Alternatively, the betamethasone (including a salt, hydrate or solvate thereof) may be substituted by at least one other corticosteroid partner, for example, 1 or 2 of such other compounds (including pharmaceutically acceptable salts, or hydrates or solvates of such compounds).

Viewed from another aspect the invention provides a pharmaceutical composition as hereinbefore defined for use in the treatment or prevention of a skin disorder such as psoriasis or dermatitis.

Viewed from another aspect the invention provides a method of treating or preventing a skin disorder such as psoriasis or dermatitis in an animal subject, for example, a mammal such as rodent (mouse, rat, rabbit), monkey (or other non-human primate), pig or other laboratory animal used as a model to study skin disorders. Another suitable mammalian subject is a patient in need thereof. In one embodiment, the invention comprises administering to said subject (e.g. a human patient) an effective amount of a pharmaceutical composition as herein before defined.

Viewed from another aspect the invention provides a method of treating, such as reducing symptoms of, or preventing a skin disorder such as psoriasis or dermatitis, in a patient in need thereof comprising administering to said patient, preferably a human, an effective amount of at least one compound of formula (I) and simultaneously, in parallel, separately or sequentially administering to said patient an effective amount of at least one compound (B) (e.g., 1, 2 or 3 of such compounds) as herein defined. In sequential administration either compound can be administered first.

Viewed from another aspect the invention provides a method of treating, such as reducing symptoms of, or preventing a skin disorder such as psoriasis or dermatitis, in a patient in need thereof comprising:
(i) identifying a patient who has received either a compound of formula (I) or a compound (B);
(ii) administering to said patient an effective amount of either at least one of a compound (B) as herein defined or at least one of a compound of formula (I) as herein before defined so that said patient is administered with both at least one compound of formula (I) and at least one compound (B).

In preferred embodiments, 1, 2 or 3 of compound B will be suitable for use with the invention with 1 or 2 of compound B being preferred for many invention applications.

Viewed from another aspect the invention provides use of a pharmaceutical composition as hereinbefore defined in the manufacture of a medicament for treating or preventing a skin disorder such as psoriasis or dermatitis.

Viewed from another aspect the invention provides a process for the preparation of a pharmaceutical composition as hereinbefore defined comprising blending at least one compound of formula (I) or a pharmaceutically acceptable salt, or a hydrate or solvate thereof and at least one compound (B) or a pharmaceutically acceptable salt, or a hydrate or solvate thereof in the presence of at least one pharmaceutical excipient.

### Definitions

In an alkyl group, these may be linear or branched, preferably linear.

In one embodiment, the invention relates to a pharmaceutical composition in which at least one compound (I) and at least one corticosteroid partner (e.g., 1, 2, or 3 of such compounds) are blended together in a single composition. The invention also relates to a pharmaceutical composition in the form of a kit in which the active compounds are provided in separate compositions but are designed for administration simultaneously, in parallel), separately or sequentially. Any method for treating or preventing a skin disorder as defined herein encompasses simultaneous, in parallel, separate or sequential administration of the active components or administration of the composition of the invention.

The pharmaceutical composition of the invention is a “combination”, which means either a fixed combination in one dosage unit form, or non fixed combination such as a kit of parts for combined administration where a compound of the formula (I) and at least one corticosteroid partner(s) (e.g., 1, 2 or 3 of such compounds) may be administered independently at the same time (e.g., in parallel) or separately within time intervals, especially where these time intervals allow that the combination partners show a cooperative and preferably a synergistic effect.

Thus a “pharmaceutical composition” as used herein means a product suitable for pharmaceutical use that results from the mixing, admixing or combining more than one active ingredient and includes both fixed and non-fixed combinations of the active ingredients. The term “fixed combination” or “fixed dose” means that the active ingredients, e.g. a compound of formula (I) and a corticosteroid partner such as betamethasone, are both administered to a patient simultaneously in the form of a single entity or dosage. The pharmaceutical composition can also be a “non-fixed combination” which means that the active ingredients, e.g. a compound of formula (I) and the combination partner betamethasone are both administered to a patient as separate entities either simultaneously, in parallel, concurrently or sequentially with no specific time limits, wherein such administration provides therapeutically effective levels of the two compounds in the body of the animal in need thereof.

A “corticosteroid partner” as used herein means a synthetic or semisynthetic corticosteroid generally suitable for intended goals of the invention. Preferred corticosteroid partners include the following: betamethasone, clobetasol, halometasone, dexamethasone, fluocortolone, desoximetasone, diflorasone, fluocinonide, flurandrenolide, halobetasol, amcinonide, halocinonide, triamcinolone, hydrocortisone, aclometasone, fluticasone, mometasone, clocortolone, fluocinolone, desonide, prednisone, prednisolone, and prednicarbate or a pharmaceutically acceptable salt, or a hydrate or solvate thereof. Betamethasone and its pharmaceutically acceptable salts, hydrates and solvates thereof are especially preferred corticosteroid partners.

All discussion below relating to preferred compounds of the invention is equally applicable to both these aspects of the invention.

### DETAILED DESCRIPTION

This invention concerns a combination therapy of a least one compound of formula (I) and at least one corticosteroid partner, in particular 1, 2 or 3 of such compounds with 1 or 2 compounds being preferred for many invention applications. In a preferred embodiment, betamethasone or a pharmaceutically acceptable salt, or a hydrate or solvate thereof is the corticosteroid partner. We have surprisingly found that this combination therapy results in synergy. Our results demonstrate a reduction in the proliferation and viability of HaCaT cells, the pharmaceutical composition offering a larger decrease than could have been expected from the use of compounds individually, i.e. the combination of the compounds produces an overall effect that is greater than the sum of the individual elements.

### Pharmaceutical Composition of the Invention

The invention relies on the therapeutic combination of at least one compound of formula (I) and at least one corticosteroid partner such as betamethasone or a pharmaceutically acceptable salt, or a hydrate or solvate thereof. The compound of formula (I) is

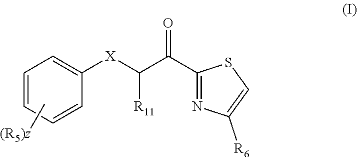

wherein X is O or S, preferably O

R₆ is H, C₁₋₆alkyl, —(CH₂)ₚCOOH, —(CH₂)ₚCOOC₁₋₆alkyl, —(CH₂)ₚCONH₂, —(CH₂)ₚCONHC₁₋₆alkyl, —(CH₂)ₚCON(C₁₋₆alkyl)₂,

R¹¹ is H or C₁₋₆ alkyl;

each R₅ is —OC₁₋₁₀alkyl, —SC₁₋₁₀alkyl, —C₁₋₁₂alkyl, or OAr²;

wherein Ar² is phenyl, optionally substituted with one or more halo;

each p is 0 to 3;

each z is 1 to 2;

or a pharmaceutically acceptable salt, or a hydrate or solvate thereof.

It is preferred if X is O.

It is preferred if R₆ is —COOC₁₋₆alkyl, or —CONHC₁₋₆alkyl, e.g. —COOC₁₋₂alkyl, or —CONHC₁₋₂alkyl.

It is preferred if R¹¹ is H or methyl, preferably H.

It is preferred if z is 1. It is preferred if p is 0.

It is preferred if the R₅ group is in the para position on the ring.

It is preferred if R₅ is —OC₄₋₁₀alkyl, —SC₄₋₁₀alkyl, —C₄₋₁₀alkyl, or OAr²;

wherein Ar² is phenyl, optionally substituted with one halo. Halo means halogen and is preferably Cl or F, especially F.

More preferably, the compound of formula (I) is

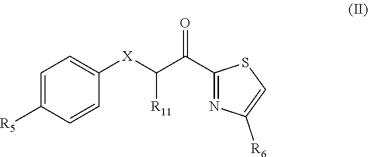

wherein X is O or S;

R₆ is H, C₁₋₆alkyl, —(CH₂)ₚCOOH, —(CH₂)ₚCOOC₁₋₆alkyl, —(CH₂)ₚCONH₂, —(CH₂)ₚCONHC₁₋₆alkyl, —(CH₂)ₚCON(C₁₋₆alkyl)₂,

R¹¹ is H or C₁₋₆ alkyl;

R₅ is —OC₁₋₁₀alkyl, —SC₁₋₁₀alkyl, —C₁₋₁₂alkyl, or OAr²;

Ar² is phenyl, optionally substituted with one or more halo;

each p is 0 to 3;

or a pharmaceutically acceptable salt, or a hydrate or solvate thereof.

More preferred compounds of the invention are those of formula (III):

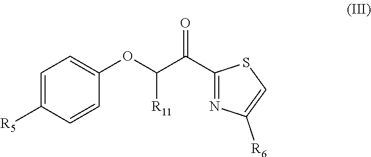

R₆ is —(CH₂)ₚCOOC₁₋₆alkyl, or —(CH₂)ₚCONHC₁₋₆alkyl;

R¹¹ is H or methyl;

R₅ is —OC₁₋₁₀alkyl, —SC₁₋₁₀alkyl, —C₁₋₁₂alkyl, or OAr²;

Ar² is phenyl, optionally substituted with one halo;

each p is 0 to 3;

or a pharmaceutically acceptable salt, or a hydrate or solvate thereof.

More preferred compounds of the invention are those of formula (IV):

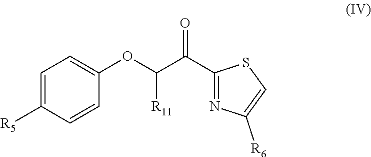

R₆ is —COOC₁₋₆alkyl, or —CONHC₁₋₆alkyl;

R¹¹ is H or methyl;

R₅ is —OC₁₋₁₀alkyl, —SC₁₋₁₀alkyl, —C₁₋₁₂alkyl, or OAr²;

Ar² is phenyl, optionally substituted with one halo;

or a pharmaceutically acceptable salt, or a hydrate or solvate thereof.

Preferred compounds are:

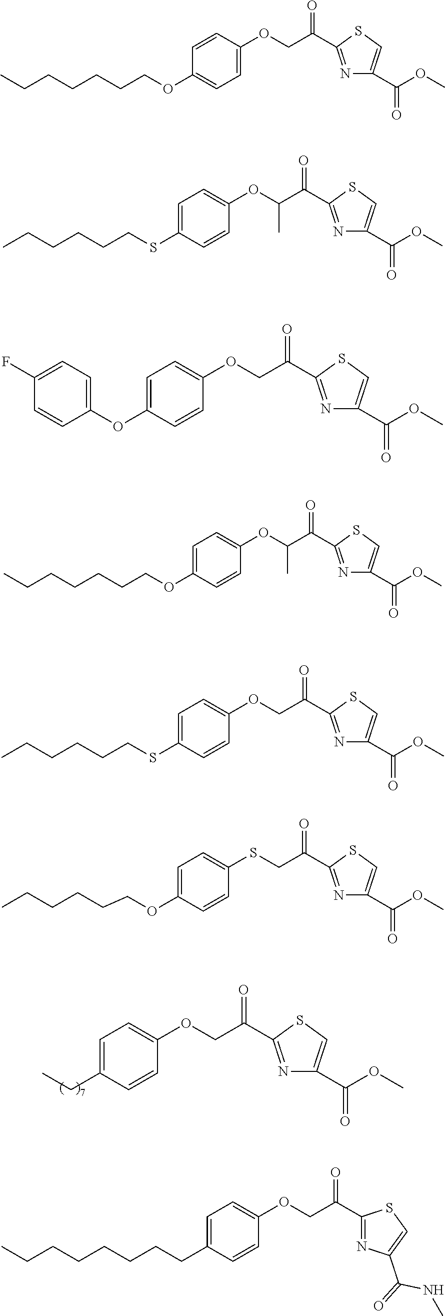

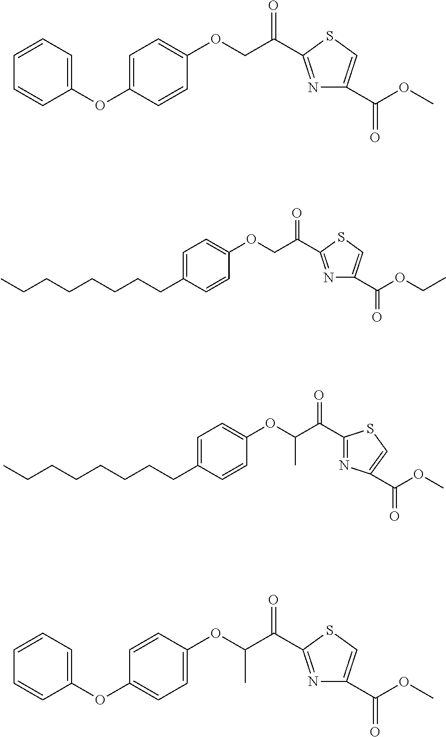

or a pharmaceutically acceptable salt, or a hydrate or solvate thereof.

Especially preferred compounds are:

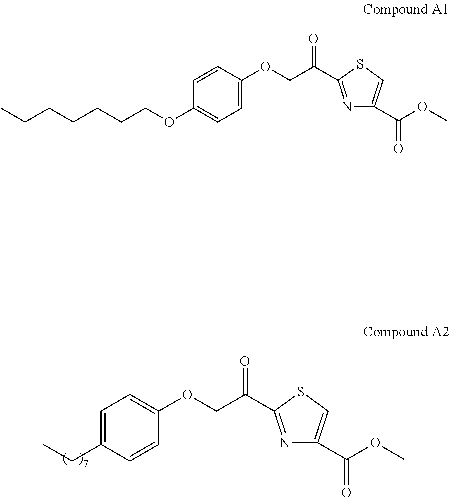

or a pharmaceutically acceptable salt, or a hydrate or solvate thereof.

It will be appreciated that the pharmaceutical composition of the invention may comprise one or more than one compound of formula (I) as herein before defined, for example, 1, 2 or 3 of such compounds with 1 or 2 compounds being preferred for most invention applications Salts, hydrates or solvates of any of these compounds can also be used.

Where possible, the compounds of the invention can be administered in salt, hydrate or solvate form, especially salt form.

Typically, a pharmaceutical acceptable salt may be readily prepared by using a desired acid. The salt may precipitate from solution and be collected by filtration or may be recovered by evaporation of the solvent. For example, an aqueous solution of an acid such as hydrochloric acid may be added to an aqueous suspension of a compound of formula (I) and the resulting mixture evaporated to dryness (lyophilised) to obtain the acid addition salt as a solid. Alternatively, a compound of formula (I) may be dissolved in a suitable solvent, for example an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

Suitable addition salts are formed from inorganic or organic acids which form non-toxic salts and examples are hydrochloride, hydrobromide, hydroiodide, sulphate, bisulphate, nitrate, phosphate, hydrogen phosphate, acetate, trifluoroacetate, maleate, malate, fumarate, lactate, tartrate, citrate, formate, gluconate, succinate, pyruvate, oxalate, oxaloacetate, trifluoroacetate, saccharate, benzoate, alkyl or aryl sulphonates (e.g. methanesulphonate, ethanesulphonate, benzenesulphonate or p-toluenesulphonate) and isethionate. Representative examples include trifluoroacetate and formate salts, for example the bis or tris trifluoroacetate salts and the mono or diformate salts, in particular the tris or bis trifluoroacetate salt and the monoformate salt.

Compounds of formula (I) may be manufactured using known chemical synthetic routes. Synthesis methods are outlined in WO2014/118195 and WO2011/039563 as well as references cited therein.

### Corticosteroid

The second component (compound B i.e. the corticosteroid partner) of the composition of the invention is a corticosteroid, preferably a synthetic or semi-synthetic corticosteroid, especially betamethasone or a pharmaceutically acceptable salt, or a hydrate or solvate thereof. Betamethasone is a compound of formula:

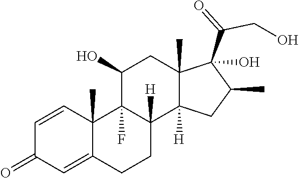

In any composition of the invention the corticosteroid may be present in a salt or non salt form. In particular, in any composition of the invention betamethasone may be present in a salt or non salt form. If a salt form is used, any conventional salt form is possible.

Betamethasone is a known commercial product and any known commercial form of betamethasone can be used. Conveniently, the salt form used is the valerate, acetate or propionate salt. The salt may be a monosalt form, disalt or trisalt form, given the presence of multiple hydroxy groups on which salts can be formed.

If a mono salt form of betamethasone is used, the salt can be present in the 17 position. A disalt form typically comprises a salt at the 17 and 21 positions of the molecule.

Suitable forms include betamethasone diproprionate, betamethasone valerate, betamethasone acetate and betamethasone sodium phosphate.

Whilst the invention is primarily described with reference to betamethasone, it is envisaged that other corticosteroids could also be combined with the compounds of formula (I) to form synergistic combinations.

Possible further corticosteroids include clobetasol, halometasone, dexamethasone, fluocortolone, desoximetasone, diflorasone, fluocinonide, flurandrenolide, halobetasol, amcinonide, halocinonide, triamcinolone, hydrocortisone, aclometasone, fluticasone, mometasone, clocortolone, fluocinolone, desonide, prednisone, prednisolone and prednicarbate.

Preferred options include Betamethasone, Clobetasol, Halobetasol, Diflorasone, Fluocinonide, Halcinonide, Amcinonide, Desoximetasone, Triamcinolone, Mometasone, Fluticasone, Halometasone, Hydrocortisone, Flurandrenolide, Desonide, Fluocinolone, and Alclometasone or a salt thereof.

Specific corticosteroid compounds include Clobetasol propionate, Betamethasone dipropionate, Halobetasol propionate, Diflorasone diacetate, Fluocinonide, Halcinonide, Amcinonide, Desoximetasone, Triamcinolone acetonide, Mometasone furoate, Fluticasone propionate, Halometasone, Fluocinolone acetonide, Hydrocortisone valerate, Hydrocortisone butyrate, Flurandrenolide, Triamcinolone acetonide, Mometasone furoate, Fluticasone propionate, Desonide, Fluocinolone acetonide, and Alclometasone dipropionate.

The use of betamethasone type corticosteroids is especially preferred such as betamethasone, dexamethasone and fluocortolone or salts thereof.

In one embodiment, the invention provides a pharmaceutical composition comprising:

(A) a compound of formula (I):

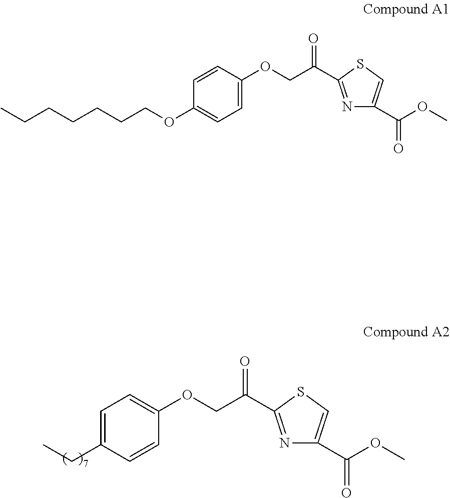

or a salt thereof and

(B) a corticosteroid partner selected from the group consisting of betamethasone, clobetasol, halometasone, dexamethasone, fluocortolone, desoximetasone, diflorasone, fluocinonide, flurandrenolide, halobetasol, amcinonide, halocinonide, triamcinolone, hydrocortisone, aclometasone, fluticasone, mometasone, clocortolone, fluocinolone, desonide, prednisone, prednisolone, and prednicarbate or a salt thereof, especially betamethasone, dexamethasone and fluocortolone or a salt thereof.

Alternatively, and as discussed above, the compositions of the invention could comprise betamethasone and additionally comprise one or more further corticosteroids (e.g., 1, 2, or 3) to augment the properties of the composition of the invention. Suitable additional corticosteroids include clobetasol, halometasone, dexamethasone, fluocortolone, desoximetasone, diflorasone, fluocinonide, flurandrenolide, halobetasol, amcinonide, halocinonide, triamcinolone, hydrocortisone, aclometasone, fluticasone, mometasone, clocortolone, fluocinolone, desonide, prednisone, prednisolone and prednicarbate or salts thereof Alternatively, one or more of the aforementioned corticosteroids could be substituted for the betamethasone (including its salts, hydrates and solvates thereof) so long as intended invention results are achieved.

It is also within the scope of the invention to combine the composition of the invention with other compounds conventionally used in conjunction with corticosteroids such as betamethasone in pharmaceuticals. For example, betamethasone can be combined with clotrimazole and optionally gentamicin. Betamethasone can be combined with salicylic acid, e.g. for use in the treatment of psoriatic skin conditions. The combination of betamethasone with calcipotriol is also a known therapy for psoriasis and hence the inclusion of calcipotriol in the compositions of the invention is envisaged.

Viewed from another aspect therefore, the invention provides a pharmaceutical composition, or kit as previously described further comprising clotrimazole, gentamicin salicylic acid, or calcipotriol or a salt, hydrate or solvate thereof.

The amounts of each compound present in the composition of the invention are determined in molar terms, and the ratio of each is preferably corticosteroid to compound (I) of 20:1 to 1:1 moles, such as 15:1 to 5:1 moles. There is therefore generally an excess of the corticosteroid in molar terms.

The amount of the compounds of the invention in the composition will often be determined by the physician depending on the dosage required.

### Skin Disorders

As noted above, the invention targets skin disorders, especially psoriasis and dermatitis. In particular, it is envisaged that the compositions of the invention may reduce inflammation and/or itchiness associated with the skin condition in question.

The combination therapy of the invention may have utility in treating a variety of different forms of dermatitis, such as atopic dermatitis or contact dermatitis. Thus, the compounds of the invention may be used to treat contact dermatitis such as allergic contact dermatitis or irritant contact dermatitis.

The nature of the allergan or irritant which causes the contact dermatitis can vary a lot and many people have different reactions to different allergans/irritants.

One of the most common causes of allergic contact dermatitis are plants of the Toxicodendron genus: poison ivy, poison oak, and poison sumac. Certain alkyl resorcinols such as bilobol found in Gingko biloba fruits are strong skin irritants. Other allergens include nickel, gold, balsam of Peru (Myroxylon pereirae), and chromium.

Common causes of irritant contact dermatitis are harsh (highly alkaline) soaps, detergents, and cleaning products. Irritant contact dermatitis can be divided into forms caused by chemical irritants and those caused by physical irritants. Common chemical irritants implicated include solvents (alcohol, xylene, turpentine, esters, acetone, ketones, and others); metalworking fluids (neat oils, water-based metalworking fluids with surfactants); latex; kerosene; ethylene oxide; surfactants in topical medications and cosmetics (sodium lauryl sulfate); alkalies (drain cleaners, strong soap with lye residues). Physical irritant contact dermatitis may most commonly be caused by low humidity from air conditioning. Also, many plants directly irritate the skin.

A further form of contact dermatitis is photocontact dermatitis. The skin condition is caused by exposure to ultraviolet light (320-400 nm UVA).

The invention may also lead to a treatment of atopic dermatitis. Atopic dermatitis is a type of eczema and is an inflammatory, chronically relapsing, non-contagious and itchy skin disorder.

Other less common forms of dermatitis to be treated include dermatitis herpetiformis, seborrheic dermatitis and stasis dermatitis.

By treating or treatment is meant at least one of:

(i). inhibiting the disease i.e. arresting, reducing or delaying the development of the disease or a relapse thereof or at least one clinical or subclinical symptom thereof, or

(ii). relieving or attenuating one or more of the clinical or subclinical symptoms of the disease.

By prevention is meant (i) preventing or delaying the appearance of clinical symptoms of the disease developing in a mammal.

The benefit to a subject to be treated is either statistically significant or at least perceptible to the patient or to the physician. In general a skilled man can appreciate when “treatment” occurs. It is particularly preferred if the pharmaceutical compositions of the invention are used therapeutically, i.e. to treat a condition which has manifested rather than prophylactically. It may be that the pharmaceutical composition of the invention is more effective when used therapeutically than prophylactically.

The pharmaceutical composition of the invention can be used on any animal subject, in particular a mammal and more particularly a human or an animal serving as a model for a disease (e.g., rat, mouse, pig, monkey, etc.). For example, in one use a pharmaceutical combination of the invention is used as a positive control in the animal subject to test other compounds for activity and/or side effects.

In order to treat a disease an effective amount of the active pharmaceutical composition needs to be administered to a patient. A “therapeutically effective amount” means the amount of a pharmaceutical composition that, when administered to an animal for treating a state, disorder or condition, is sufficient to effect such treatment. The “therapeutically effective amount” will vary depending on the pharmaceutical composition, the disease and its severity and the age, weight, physical condition and responsiveness of the subject to be treated and will be ultimately at the discretion of the attendant doctor.

It may be that to treat skin disorders according to the invention that the pharmaceutical composition of the invention has to be readministered at certain intervals. Suitable dosage regimes can be prescribed by a physician.

The pharmaceutical composition of the invention typically comprises the active components in admixture with at least one pharmaceutically acceptable carrier selected with regard to the intended route of administration and standard pharmaceutical practice.

The term “carrier” refers to a diluent, excipient, and/or vehicle with which an active compound is administered. The pharmaceutical compositions of the invention may contain combinations of more than one carrier. Such pharmaceutical carriers are well known in the art. The pharmaceutical compositions may also comprise any suitable binder(s), lubricant(s), suspending agent(s), coating agent(s), and/or solubilizing agent(s) and so on. The pharmaceutical composition can also contain other active components, e.g. other drugs for the treatment of skin disorders.

It will be appreciated that pharmaceutical compositions for use in accordance with the present invention may be in the form of oral, parenteral, transdermal, sublingual, topical, implant, nasal, or enterally administered (or other mucosally administered) suspensions, capsules or tablets, which may be formulated in conventional manner using one or more pharmaceutically acceptable carriers or excipients. The pharmaceutical compositions of the invention could also be formulated as nanoparticle formulations.

However, for the treatment of skin disorders, the pharmaceutical composition of the invention will preferably be administered topically. The pharmaceutical composition may therefore be provided in the form of a cream, gel, foam, salve or ointment.

The pharmaceutical composition of the invention may contain from 0.01 to 99% weight—per volume of the active material. The therapeutic doses will generally be between about 10 and 2000 mg/day and preferably between about 30 and 1500 mg/day of active components combined. Other ranges may be used, including, for example, 50-500 mg/day, 50-300 mg/day, 100-200 mg/day or active components combined.

Administration may be once a day, twice a day, or more often, and may be decreased during a maintenance phase of the disease or disorder, e.g. once every second or third day instead of every day or twice a day. The dose and the administration frequency will depend on the clinical signs, which confirm maintenance of the remission phase, with the reduction or absence of at least one or more preferably more than one clinical signs of the acute phase known to the person skilled in the art.

The invention is described further below with reference to the following non-limiting examples and figures.

### DESCRIPTION OF FIGURES

FIG. 1a-c: Dose response of Compounds A1 and A2, and Betamethasone on immortalized keratinocyte cell line HaCat cell viability. Data presented are average and standard deviation of 3 independent experiments performed in series of 8 technical replicates per treatment. Star (*) represent significant difference compare to control (100%) (*P≤0.05; **P≤0.01; ***P≤0.001; ****P≤0.0001).

FIG. 2: Co-treatment with cPLA2α inhibitors Compounds A1 and A2 and corticosteroid hormone receptor agonist Betamethasone shows synergistic effects on human Keratinocyte cell viability compared to each inhibitor alone. Data presented are average and standard deviation of 3 independent experiments performed in series of 8 technical replicates per treatment. Star (*) represent significant difference in compare to control (100%) (*P≤0.05; **P≤0.01; ***P≤0.001; ****P≤0.0001).

### EXAMPLE 1

### Methods: Cell Culture:

The spontaneously immortalized, nontumorigenic skin keratinocyte cell line HaCaT was maintained in DMEM supplemented with 5% (v/v) FBS, 0.3 mg/ml glutamine and 0.1 mg/ml gentamicin at 37° C. with 5% CO₂ in a humidified atmosphere. Subculture using trypsin-EDTA was performed every 3-4 days with split ratio of 1:3-1:4 to ensure actively proliferating cells.

### Resazurin Assay:

Cells were seeded in 96 well plates in fully supplemented medium at a density of 3000 cells per well. Following 72 hour of cultivation, the cells were starved of serum in 0.25% FBS/DMEM overnight to halt proliferation, synchronize the cells and to increase cell sensitivity to treatment. Next day, the cells were treated with cPLA2α inhibitor Compound B, Compound A, vitamin D analogue Calcipotriol and corticosteroid hormone receptor agonist Betamethasone dipropionate for 24 hours. Resazurin was added next day according to the manufacturer's instruction (RnD Systems, UK) and left to incubate for 2 hour in incubator at 37° C. with 5% CO₂ in a humidified atmosphere before fluorescence was read at 544 nm excitation and 590 nm emission wavelength. The cells were observed under the microscope to evaluate possible morphology changes and signs of stress before addition of resazurin. The experiments were performed in series of 8 wells per treatment and repeated 3 times.

The following compounds are used:

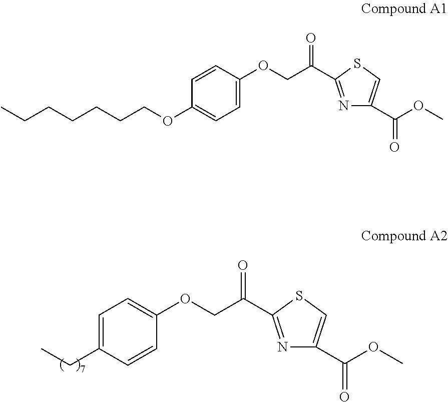

### Results:

cPLA2α Inhibitor Compounds A1 and A2, and Corticosteroid Hormone Receptor Agonist Betamethasone Shows Dose Response on Immortalized Keratinocyte Cell Line Hacat Cell Viability.

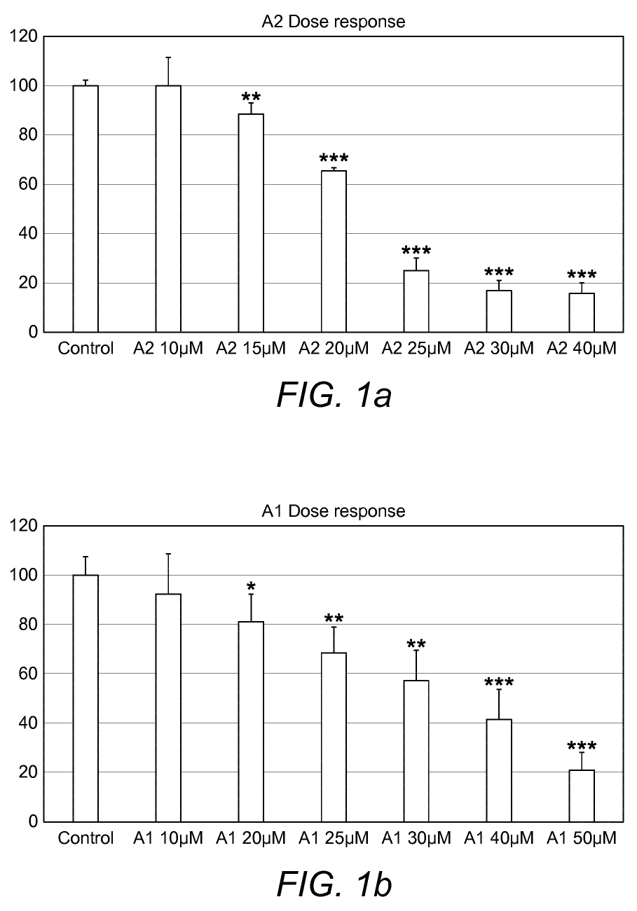

It has been shown before that Betamethasone dipropionate effectively halt proliferation of Hacat keratinocytes. On the basis of these results, the combinatorial effect of Betamethasone has been proposed to use in the treatment of Psoriasis. In this study, to reconfirm the previous outcome, experiments were performed to determine dose response of Betamethasone dipropionate. In addition, dose response of therapeutic molecules (A1 and A2), inhibitors of cPLA2α, has also been tested for the first time on Hacat keratinocytes proliferation study. According to the results in the experiment, Hacat keratinocytes shows resistance to Betamethasone up to 200 μM (FIG. 1c). The little effect seen is rather because of higher concentration of solvent DMSO than Betamethasone (FIG. 1c).

Co-Treatment with cPLA2α Inhibitor Compound A2 and Corticosteroid Hormone Receptor Agonist Betamethasone Shows Synergistic Effects on Immortalized Keratinocyte Cell Line Hacat Cell Viability Compared to Each Inhibitor Alone.

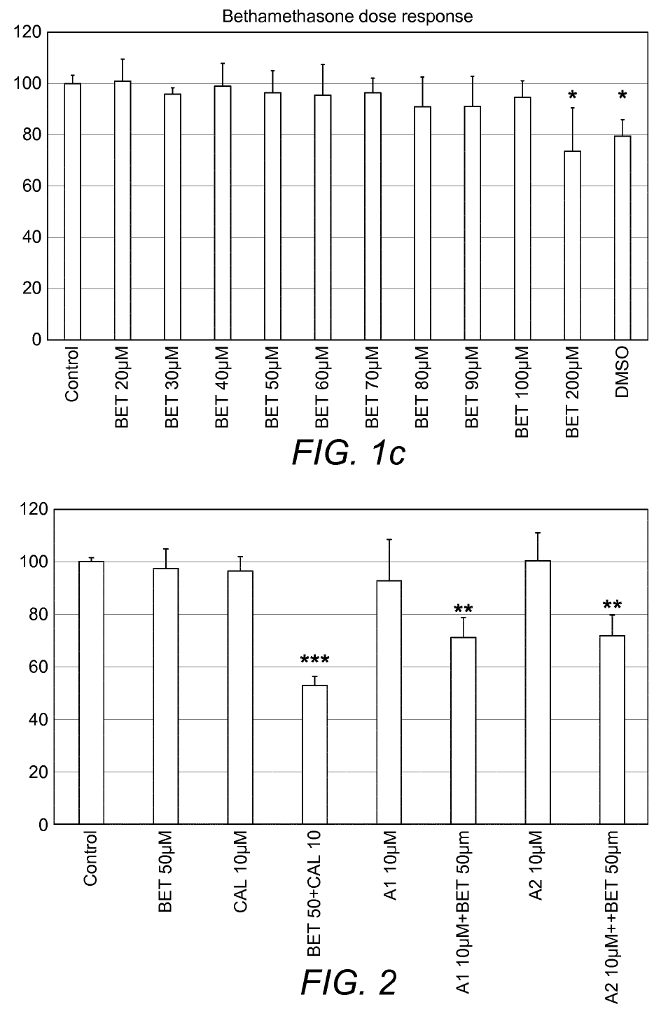

Initial experiments were performed to determine dose response of Compound A2 and Betamethasone alone (FIG. 1). The suboptimal dose of reduced viability found in inhibitor Compound A2 and Betamethasone was 10 μM and 50 μM, respectively (FIG. 1a). Combination of the inhibitor Compound A2 and receptor agonist Betamethasone were also compared with already established combo of Betamethasone and Calcipotriol. Following 24 hours of treatment, sub-toxic doses of Betamethasone (50 μM) and Calciptoriol (10 μM) shows 45% reduction in proliferation of HaCat cells. The combination of Compound A2 10 μM with Betamethasone 50 μM shows 25% reduction (FIG. 2).

This observed trend of synergistic effects on cell viability reduction indicate beneficial effects of co-treatment of Compound A1 and Betamethasone on skin disorders.

Co-Treatment with cPLA2α Inhibitor Compound A1 and Corticosteroid Hormone Receptor Agonist Betamethasone Shows Synergistic Effects on Immortalized Keratinocyte Cell Line Hacat Cell Viability Compared to Each Inhibitor Alone.

Initial experiments were performed to determine dose response of Compound A1 and Betamethasone alone (FIG. 1). The suboptimal dose of reduced cell viability found in inhibitor Compound A1 and Betamethasone was 10 μM and 50 μM, respectively (FIG. 1). Combination of the inhibitor Compound A1 and receptor agonist Betamethasone were also compared with already established combo of Betamethasone and Calcipotriol. Following 24 hours of treatment, sub-toxic doses of Betamethasone (50 μM) and Calciptoriol (10 μM) shows 45% reduction in proliferation of HaCat cells (FIG. 2). The combination of Compound A1 10 μM with Betamethasone 50 μM shows 25% reduction This observed trend of synergistic effects on cell viability reduction indicate beneficial effects of co-treatment of Compound A1 and Betamethasone on skin disorders.

1. A pharmaceutical composition comprising:
(A) at least one compound of formula (I): 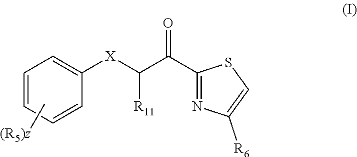
wherein X is O or S, preferably O
R₆ is H, C₁₋₆alkyl, —(CH₂)ₚCOOH, —(CH₂)ₚCOOC₁₋₆alkyl, —(CH₂)ₚCONH₂, —(CH₂)ₚCONHC₁₋₆alkyl, —(CH₂)ₚCON(C₁₋₆alkyl)₂,
R¹¹ is H or C₁₋₆ alkyl;
each R₅ is —OC₁₋₁₀alkyl, —SC₁₋₁₀alkyl, —C₁₋₁₂alkyl, or OAr²;
wherein Ar² is phenyl, optionally substituted with one or more halo;
each p is 0 to 3;
each z is 1 to 2;
or a pharmaceutically acceptable salt, or a hydrate or solvate thereof; and
(B) one or more corticosteroid partners, preferably selected from the group consisting of betamethasone, clobetasol, halometasone, dexamethasone, fluocortolone, desoximetasone, diflorasone, fluocinonide, flurandrenolide, halobetasol, amcinonide, halocinonide, triamcinolone, hydrocortisone, aclometasone, fluticasone, mometasone, clocortolone, fluocinolone, desonide, prednisone, prednisolone,and prednicarbate or a pharmaceutically acceptable salt, or a hydrate or solvate thereof, especially betamethasone or a pharmaceutically acceptable salt, or a hydrate or solvate thereof.

2. The pharmaceutical composition of claim 1 wherein the pharmaceutical composition is a fixed combination or non-fixed combination.

3. The pharmaceutical composition as claimed in claim 1 for simultaneous, parallel, sequential or separate use comprising a kit comprising a first composition comprising at least one compound of formula (I) as defined in claim 1 and a pharmaceutically-acceptable diluent or carrier, and a second composition comprising at least one compound (B) as defined in claim 1 and a pharmaceutically-acceptable diluent or carrier.

4. The pharmaceutical composition as claimed in claim 1 wherein the compound (B) is betamethasone, dexamethasone or fluocortolone or a pharmaceutically acceptable salt, or a hydrate or solvate thereof.

5. The pharmaceutical composition as claimed in claim 1 wherein the compound (B) is betamethasone or a pharmaceutically acceptable salt, or a hydrate or solvate thereof.

6. The pharmaceutical composition as claimed in claim 1 wherein the compound (B) is betamethasone diproprionate, betamethasone valerate, betamethasone acetate or betamethasone sodium phosphate.

7. The pharmaceutical composition as claimed in claim 1 wherein the compound of formula (I) is represented by formula (II): 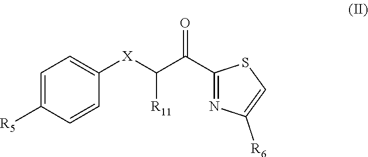
wherein X is O or S;
R₆ is H, C₁₋₆alkyl, —(CH₂)ₚCOOH, —(CH₂)ₚCOOC₁₋₆alkyl, —(CH₂)ₚCONH₂, —(CH₂)ₚCONHC₁₋₆alkyl, —(CH₂)ₚCON(C₁₋₆alkyl)₂,
R¹¹ is H or C₁₋₆ alkyl;
R₅ is —OC₁₋₁₀alkyl, —SC₁₋₁₀alkyl, —C₁₋₁₂alkyl, or OAr²;
Ar² is phenyl, optionally substituted with one or more halo;
each p is 0 to 3;
or a pharmaceutically acceptable salt, or a hydrate or solvate thereof.

8. The pharmaceutical composition as claimed in claim 1 wherein the compound of formula (I) is represented by formula (III): 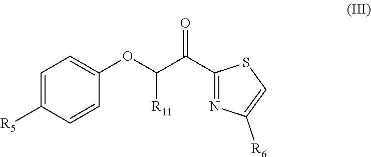
R₆ is —(CH₂)ₚCOOC₁₋₆alkyl, or —(CH₂)ₚCONHC₁₋₆alkyl;
R¹¹ is H or methyl;
R₅ is —OC₁₋₁₀alkyl, —SC₁₋₁₀alkyl, —C₁₋₁₂alkyl, or OAr²;
Ar² is phenyl, optionally substituted with one halo;
each p is 0 to 3;
or a pharmaceutically acceptable salt, or a hydrate or solvate thereof.

9. The pharmaceutical composition as claimed in claim 1 wherein the compound of formula (I) is represented by formula (IV): 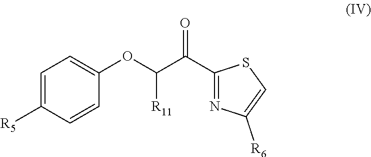
R₆ is —COOC₁₋₆alkyl, or —CONHC₁₋₆alkyl;
R¹¹ is H or methyl;
R₅ is —OC₁₋₁₀alkyl, —SC₁₋₁₀alkyl, —C₁₋₁₂alkyl, or OAr²;
Ar² is phenyl, optionally substituted with one halo;
or a pharmaceutically acceptable salt, or a hydrate or solvate thereof.

10. The pharmaceutical composition as claimed in claim 1 wherein said compound of formula (I) is 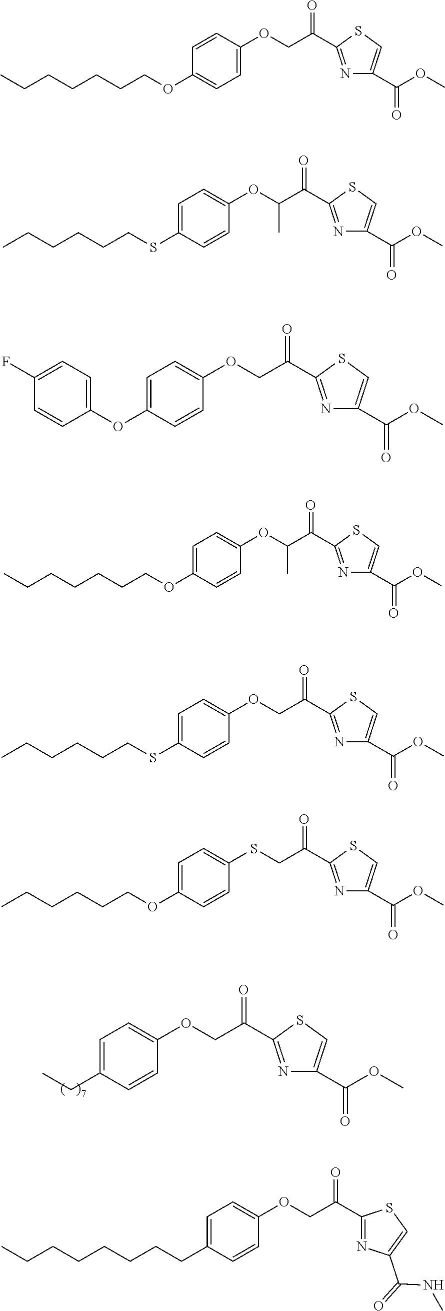 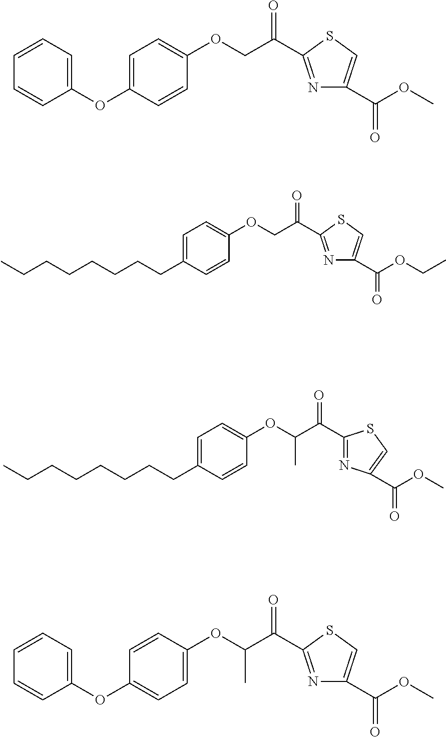
or a pharmaceutically acceptable salt, or a hydrate or solvate thereof.

11. The pharmaceutical composition as claimed in claim 1 where the compound of formula (I) is Compound A1 or Compound A2: 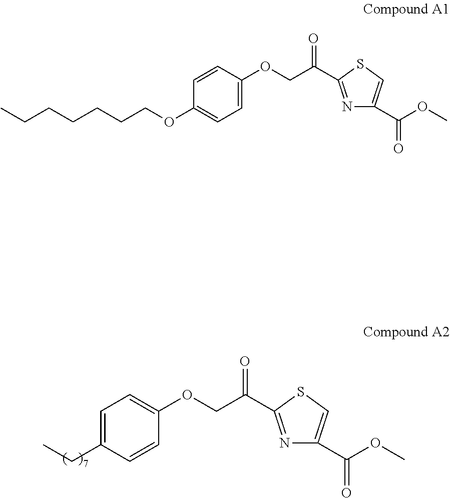
or a salt thereof.

12. The pharmaceutical composition as claimed in claim 1 wherein the molar ratio of compound of formula (I) to compound (B) in the pharmaceutical composition is 1:1 to 1:20.

13. The pharmaceutical composition as claimed in claim 1 further comprising clotrimazole, gentamicin, salicylic acid or calcipotriol or a pharmaceutically acceptable salt, or a hydrate or solvate thereof.

14. (canceled)

15. A method of treating, such as reducing symptoms of, or preventing a skin disorder such as psoriasis or dermatitis in a patient in need thereof comprising administering to said patient, preferably a human, an effective amount of a pharmaceutical composition as claimed in claim 1.

16. A method of treating, such as reducing symptoms of, or preventing a skin disorder such as psoriasis or dermatitis in a patient in need thereof comprising administering to said patient, preferably a human, an effective amount of at least one compound of formula (I) as defined in claim 1 and simultaneously, in parallel, separately or sequentially administering to said patient at least one compound (B) as defined in claim 1.

17. A method of treating such as, reducing symptoms of, or preventing a skin disorder such as psoriasis or dermatitis, in a patient in need thereof comprising:
identifying a patient who has received either a compound of formula (I) as or a compound (B) as defined in claim 1; and
(ii) administering to said patient an effective amount of either at least one compound (B) or at least one compound of formula (I) as defined in claim 1 so that said patient is administered with both a compound of formula (I) and a compound (B).

18. A method of treating, such as reducing symptoms of, or preventing a skin disorder such as psoriasis or dermatitis in an animal subject in need thereof comprising administering to said animal an effective amount of a pharmaceutical composition as claimed in claim 1.

19. A method of treating, such as reducing symptoms of, or preventing a skin disorder such as psoriasis or dermatitis in an animal subject in need thereof comprising administering to said animal an effective amount of at least one compound of formula (I) as defined in claim 1 and simultaneously, in parallel, separately or sequentially administering to said animal at least one compound (B) as defined in claim 1.

20. The method of claim 18, wherein the animal subject is a rodent, monkey, or a pig.

21. The method of claim 19, wherein the pharmaceutical composition or the effective amount of compound of Formula I formula (I) and compound (B) is used as a positive control.

22. (canceled)

23. The pharmaceutical composition of claim 1 comprising betamethasone or a pharmaceutically acceptable salt, or a hydrate or solvate thereof optionally in combination with one or more additional corticosteroids or a pharmaceutically acceptable salt, or a hydrate or solvate thereof.

24. The pharmaceutical composition as claimed in claim 23, wherein the additional corticosteroid is selected from the group consisting of clobetasol, halometasone, dexamethasone, fluocortolone, desoximetasone, diflorasone, fluocinonide, flurandrenolide, halobetasol, amcinonide, halocinonide, triamcinolone, hydrocortisone, aclometasone, fluticasone, mometasone, clocortolone, fluocinolone, desonide, prednisone, prednisolone, and prednicarbate or a pharmaceutically acceptable salt, or a hydrate or solvate thereof.

25. The pharmaceutical composition as claimed in claim 1 in a form suitable for topical administration, e.g. a cream, gel, foam or ointment.
